(19) United States
(12) Patent Application Publication
(10) Pub. No.: US 2014/0187964 A1
(43) Pub. Date: Jul. 3, 2014

(54) Intravascular Ultrasound Catheter for Minimizing Image Distortion

(71) Applicant: Volcano Corporation, San Diego, CA (US)

(72) Inventors: Paul Douglas Corl, Palo Alto, CA (US); Dylan Van Hoven, Oceanside, CA (US)

(73) Assignee: Volcano Corporation, San Diego, CA (US)

(21) Appl. No.: 14/137,337

(22) Filed: Dec. 20, 2013

Related U.S. Application Data
(60) Provisional application No. 61/746,958, filed on Dec. 28, 2012.

(51) Int. Cl. A61B 8/00

(52) U.S. Cl. CPC A61B 8/445
USPC 600/467

(57) ABSTRACT

The present disclosure provides various embodiments of an ultrasound catheter for use in intravascular ultrasound (IVUS) imaging. An exemplary IVUS device includes an flexible elongate member having a lumen extending therethrough, and a rotatable imaging core disposed within the lumen. The imaging core is further configured to transmit and receive ultrasound signals through a distal portion of the flexible elongate member. The distal portion of the flexible elongate member includes a first set of material layers, while the proximal portion includes a second set of material layers different than the first set of material layers. The first set of material layers and the second material layers minimize distortion of the ultrasound signals. The first set of material layers further facilitates an average speed of sound through the first set of material layers that is substantially equivalent to a speed of sound through blood.

### CROSS-REFERENCE TO RELATED APPLICATIONS

The present application claims priority to and the benefit of U.S. Provisional Patent Application No. 61/746,958, filed Dec. 28, 2012, which is hereby incorporated by reference in its entirety.

### TECHNICAL FIELD

The present disclosure relates generally to intravascular ultrasound (IVUS) imaging devices, systems, and methods, and in particular, to IVUS catheters used for IVUS imaging in IVUS devices, systems, and methods.

### BACKGROUND

Intravascular ultrasound (IVUS) imaging is widely used in interventional cardiology as a diagnostic tool for assessing a vessel, such as an artery, within the human body to determine the need for treatment, to guide intervention, and/or to assess its effectiveness. An IVUS imaging system uses ultrasound echoes to form a cross-sectional image of the vessel of interest. Typically, IVUS imaging uses a transducer on an IVUS catheter that both emits ultrasound signals (waves) and receives the reflected ultrasound signals. The emitted ultrasound signals (often referred to as ultrasound pulses) pass easily through most tissues and blood, but they are partially reflected by discontinuities arising from tissue structures (such as the various layers of the vessel wall), red blood cells, and other features of interest. The IVUS imaging system, which is connected to the IVUS catheter by way of a patient interface module, processes the received ultrasound signals (often referred to as ultrasound echoes) to produce a cross-sectional image of the vessel where the IVUS catheter is located.

There are primarily two types of IVUS catheters in common use today: solid-state and rotational. An exemplary solid-state IVUS catheter uses an array of transducers (typically 64) distributed around a circumference of the catheter and connected to an electronic multiplexer circuit. The multiplexer circuit selects transducers from the array for transmitting ultrasound signals and receiving reflected ultrasound signals. By stepping through a sequence of transmit-receive transducer pairs, the solid-state IVUS catheter can synthesize the effect of a mechanically scanned transducer element, but without moving parts. Since there is no rotating mechanical element, the transducer array can be placed in direct contact with blood and vessel tissue with minimal risk of vessel trauma, and the solid-state scanner can be wired directly to the IVUS imaging system with a simple electrical cable and a standard detachable electrical connector.

An exemplary rotational IVUS catheter includes a single transducer located at a tip of a flexible driveshaft that spins inside a sheath inserted into the vessel of interest. The transducer is typically oriented such that the ultrasound signals propagate generally perpendicular to an axis of the IVUS catheter. In the typical rotational IVUS catheter, a fluid-filled (e.g., saline-filled) sheath protects the vessel tissue from the spinning transducer and driveshaft while permitting ultrasound signals to freely propagate from the transducer into the tissue and back. As the driveshaft rotates (e.g., at 30 revolutions per second), the transducer is periodically excited with a high voltage electrical pulse to emit a short burst of ultrasound. The ultrasound signals are emitted from the transducer and propagate through the fluid-filled sheath and sheath wall, in a direction generally perpendicular to an axis of rotation of the driveshaft. The same transducer then listens for returning ultrasound echo signals reflected from various tissue structures, and the IVUS imaging system assembles a two dimensional image of the vessel cross-section from a sequence of several hundred of these ultrasound pulse/echo acquisition sequences occurring during a single revolution of the transducer.

While the sheaths of conventional rotational IVUS catheter designs have been sufficient for traditional PZT ultrasound transducers, it has been found the conventional sheaths fail to adequately minimize ultrasound signal distortion, while also providing sufficient strength and flexibility, for more advanced ultrasound transducer technologies, such as piezoelectric micromachined ultrasonic transducers (PMUT) that allow for focusing of the ultrasound beam and/or single crystal composite ultrasound transducers. Accordingly, there remains a need for improved ultrasound catheters for use in IVUS imaging and associated devices, systems, and methods of manufacturing.

### SUMMARY

The present disclosure provides various embodiments of an ultrasound catheter for use in rotational intravascular ultrasound (IVUS) imaging.

An exemplary IVUS device includes a flexible elongate member having a lumen extending therethrough, and an imaging core disposed within the lumen and configured to rotate within the lumen. The flexible elongate member has a proximal portion coupled with a distal portion, and the imaging core is further configured to transmit and receive ultrasound signals through the distal portion of the flexible elongate member. The distal portion of the flexible elongate member includes a first set of material layers, and the proximal portion includes a second set of material layers different from the first set of material layers. The first set of material layers and the second set of material layers are designed to minimize friction between the driveshaft and sheath to ensure smooth rotation of the imaging core. The first set of material layers is further designed to provide an average speed of sound through the first set of material layers that is substantially equivalent to a speed of sound through blood, in addition to satisfying other requirements. In some implementations, the sheath of a rotational IVUS catheter using an advanced technology focused ultrasound transducer, such as PMUT, according to the present disclosure satisfies a number of desired operational parameters, such as providing a low friction inner surface to ensure smooth rotation of spinning driveshaft, providing sufficient column strength to allow the sheath to be advanced through the vasculature without collapsing, minimizing the attenuation, reflection, and mode conversion of the ultrasound signals to permit the ultrasound signals to freely propagate from the transducer out into the tissue and back to the transducer. Further, the sheath materials also match, as closely as feasible, the speed of sound in saline and blood to minimize the refraction of the ultrasound beam as it emerges from or returns to the transducer in order to avoid distortion/degradation of the ultrasound beam that could result in a blurrier image compared to the potential for the advanced technology focused rotational IVUS imaging catheter. An interface module may be coupled with the proximal end of the flexible elongate member, and an image processing component may be in communication with the interface module.

Both the foregoing general description and the following detailed description are exemplary and explanatory in nature and are intended to provide an understanding of the present disclosure without limiting the scope of the present disclosure. In that regard, additional aspects, features, and advantages of the present disclosure will become apparent to one skilled in the art from the following detailed description.

### BRIEF DESCRIPTIONS OF THE DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion. In addition, the present disclosure may repeat reference numerals and/or letters in the various examples. This repetition is for the purpose of simplicity and clarity and does not in itself dictate a relationship between the various embodiments and/or configurations discussed.

FIG. 1 is a schematic illustration of an intravascular ultrasound (IVUS) imaging system according to various aspects of the present disclosure.

FIG. 2A is a diagrammatic cross-sectional view of a proximal portion of an IVUS catheter of the IVUS imaging system taken along line 2A-2A in FIG. 1 according to various aspects of the present disclosure.

FIG. 2B is a diagrammatic cross-sectional view of a distal portion of the IVUS catheter of the IVUS imaging system taken along line 2B-2B in FIG. 1 according to various aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

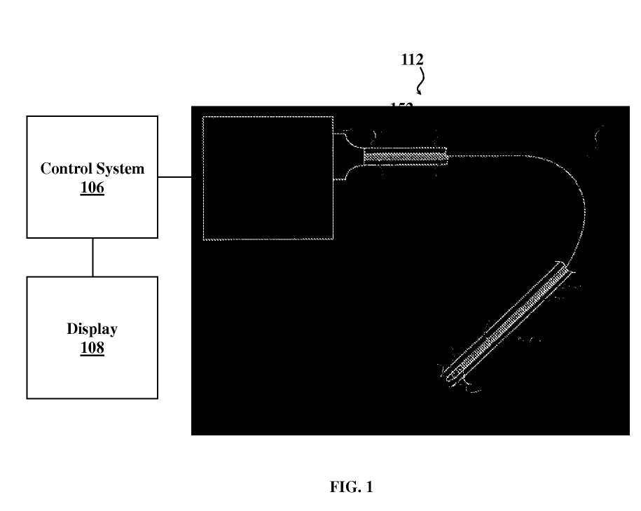

FIG. 1 is a schematic illustration of an intravascular ultrasound (IVUS) imaging system 100 according to various aspects of the present disclosure. The IVUS imaging system 100 uses ultrasound signals to generate cross-sectional images of vasculature, for example, a blood vessel, such as an artery. FIG. 1 has been simplified for the sake of clarity to better understand the inventive concepts of the present disclosure. Additional features can be added in the IVUS system 100, and some of the features described below can be replaced or eliminated for additional embodiments of the IVUS imaging system 100.

The IVUS imaging system 100 includes an IVUS catheter 102 coupled by a patient interface module (PIM) 104 to an IVUS control system 106. The control system 106 is coupled to a monitor 108 that displays an IVUS image (an image generated by the IVUS system 100), such as an image of a blood vessel in the human body. In some embodiments, wires associated with the IVUS imaging system 100 extend from the control system 106 to the interface module 104 such that signals from the control system 106 can be communicated to the interface module 104 and/or vice versa. In some embodiments, the control system 106 communicates wirelessly with the interface module 104. Similarly, in some embodiments, wires associated with the IVUS imaging system 100 extend from the control system 106 to the monitor 108 such that signals from the control system 106 can be communicated to the monitor 108 and/or vice versa. In some embodiments, the control system 106 communicates wirelessly with the monitor 108.

The IVUS catheter 102 is a rotational IVUS catheter, which may be similar to a Revolution® Rotational IVUS Imaging Catheter available from Volcano Corporation and/or rotational IVUS catheters disclosed in U.S. Pat. No. 5,243,988 and U.S. Pat. No. 5,546,948, both of which are incorporated herein by reference in their entirety. The catheter 102 is flexible such that it can adapt to the curvature of a blood vessel during use. In that regard, the curved configuration illustrated in FIG. 1 is for exemplary purposes and in no way limits the manner in which the catheter 102 may curve in other embodiments. Generally, the catheter 102 may be configured to take on any straight, arcuate, or other desired profile when in use. The catheter 102 includes an elongated, flexible catheter sheath (member) 110 (having a proximal portion 112 with a proximal end portion 114 and a distal portion 116 with a distal end portion 118) shaped and configured for insertion into a lumen of a blood vessel (not shown).

A rotating imaging core 120 extends within a lumen 122 of the catheter sheath 110. The imaging core 120 has a proximal end portion 124 disposed within the proximal end portion 114 of the catheter sheath 110 and a distal end portion 126 disposed within the distal end portion 118 of the catheter sheath 110. The distal end portion 118 of the catheter sheath 110 and the distal end portion 126 of the imaging core 120 are inserted together into a blood vessel of interest during operation of the IVUS imaging system 100. The usable length of the catheter 102 (for example, the portion that can be inserted into a patient, including the vessel of interest) can be any suitable length and can vary depending upon the application. The proximal end portion 114 of the catheter sheath 110 and the proximal end portion 124 of the imaging core 120 are connected to the interface module 104. The proximal end portions 114 and 124 are fitted with a catheter hub 130 that is removably connected to the interface module 104. The catheter hub 130 facilitates and supports a rotational interface that provides electrical and mechanical coupling between the catheter 102 and the interface module 104.

The distal end portion 126 of the imaging core 120 includes a transducer assembly 140. The transducer assembly 140 is configured to rotate (either by use of a motor or other rotary device or manually by hand) to obtain images of the vessel by transmitting and receiving ultrasound signals through the distal portion 116 of the catheter sheath 110. The transducer assembly 140 can be of any suitable type for visualizing the vessel and, in particular, a stenosis in the vessel. In the depicted embodiment, the transducer assembly 140 includes a piezoelectric micromachined ultrasonic transducer (“PMUT”) transducer and associated application-specific integrated circuit (ASIC), including those transducer assemblies disclosed in U.S. Provisional Patent Application No. 61/646,062, filed May 11, 2012, titled “CIRCUIT ARCHITECTURES AND ELECTRICAL INTERFACES FOR ROTATIONAL INTRAVASCULAR ULTRASOUND (IVUS) DEVICES,” which is hereby incorporated by reference in its entirety. In other embodiments, the ultrasound transducer assembly 140 includes a focused ultrasound transducer assembly as disclosed in U.S. Provisional Patent Application No. 61/745,425, filed Dec. 21, 2012, titled “FOCUSED ROTATIONAL IVUS TRANSDUCER USING SINGLE CRYSTAL COMPOSITE MATERIAL,” which is hereby incorporated by reference in its entirety. The transducer assembly 140 may include a housing having the PMUT transducer and associated circuitry disposed therein, where the housing has an opening that ultrasound signals generated by the PMUT transducer travel through. Alternatively, the transducer assembly 140 includes a capacitive micromachined ultrasonic transducer (“CMUT”). Generally speaking, the concepts of the present disclosure can be applied to a wide array of imaging energy sources or emission protocols, including sound and/or light-based energy sources. In some implementations, at least the distal portions of the sheaths of the present disclosure are configured to facilitate use of focused energy beams such that distortion of the beam is minimized. This allows the benefits of improved imaging techniques, including the associated imaging processing, available with focused beam energy sources to be fully realized.

Rotation of the imaging core 120 (and thus rotation of the transducer assembly 140) within the catheter sheath 110 is controlled by the interface module 104, which provides user interface controls that can be manipulated by a user. The interface module 104 can receive, analyze, and/or display information received from the transducer assembly 140 through the imaging core 120. It will be appreciated that any suitable functionality, controls, information processing and analysis, and display can be incorporated into the interface module 104. In an example, the interface module 104 receives data corresponding to the ultrasound signals (echoes) detected by the imaging core 120 and forwards the received echo data to the control system 106. In an example, the interface module 104 performs preliminary processing of the echo data prior to transmitting the echo data to the control system 106. The interface module 104 may perform amplification, filtering, and/or aggregating of the echo data. The interface module 104 can also supply high- and low-voltage DC power to support operation of the catheter 102 including the circuitry within the transducer assembly 140.

As the imaging core 120, particularly the transducer assembly 140, is rotated through each revolution, the transducer assembly 140 emits ultrasound signals (pulses) at different angles and receives ultrasound signals (echoes) reflected from various structures of the vessel of interest. The received ultrasound signals provide radial image vectors (lines) that the interface module 104 and/or the control system 106 assemble into a cross-sectional image of the vessel. When generating the cross-sectional image, the interface module 104 and/or the control system 106 assume that the transducer assembly 140 received the radial image vectors at evenly spaced angles within the vessel of interest, which means the transducer assembly 140 is rotated at a uniform angular velocity within the catheter sheath 110. However, ultrasound signal distortion results from the speed of sound of through the sheath being different than the speed of sound through blood and/or the fluid filling the sheath. In particular, beam distortions and mode conversions are caused by diffraction, while the strength of the ultrasound signals is degraded due to reflections resulting from the mismatches in the acoustic properties of the sheath material(s) and the surrounding fluids. In this context, mode conversion is understood to be the transition between longitudinal and sheer waves. Only longitudinal waves are present in liquids, but sheer and longitudinal waves are present in solids (such as the sheath). Because longitudinal waves and sheer waves travel at different velocities through the material (sheer waves travel slower and have greater attenuation), the mode conversions can result in a single ultrasound ray being split into two or more rays, which necessarily leads to beam distortion, especially where a focused ultrasound beam is desired. The distorted ultrasound signals resulting from the sheath lead to undesirable image distortion.

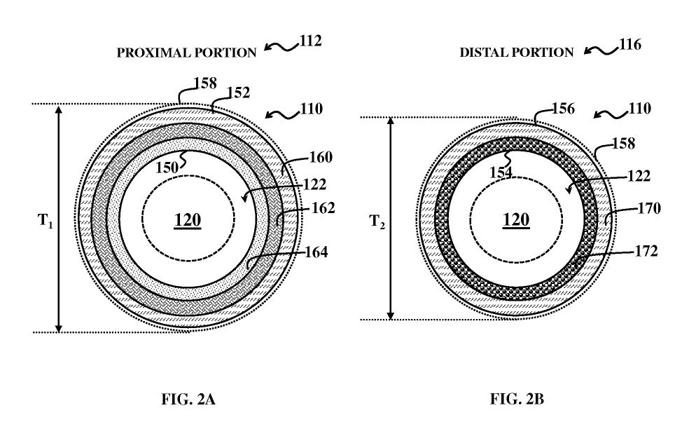

The present disclosure provides the catheter sheath 110 with a design that optimizes mechanical, chemical, and acoustic properties of the catheter 102, such that the catheter sheath 110 provides necessary structural support while minimizing beam distortion resulting from the acoustic properties of the material through which the ultrasound signals travel to facilitate use of more advanced ultrasound imaging techniques. More specifically, the proximal portion 112 of the catheter sheath 110 includes a different set of material layers than the distal portion 116 of the catheter sheath 110, such that the catheter sheath 110 minimizes image distortion, including ultrasound signal distortion. FIG. 2A is a diagrammatic cross-sectional view of the proximal portion 112 of the catheter 102, particularly the catheter sheath 110, taken along line 2A-2A in FIG. 1 according to various aspects of the present disclosure; and FIG. 2B is a diagrammatic cross-sectional view of the distal portion 116 of the catheter 102, particularly the catheter sheath 110, taken along line 2B-2B in FIG. 1 according to various aspects of the present disclosure. FIG. 2A and FIG. 2B have been simplified for the sake of clarity to better understand the inventive concepts of the present disclosure. In that regard, it is understood that additional material layers can be added to the catheter sheath 110 and/or one or more of the material layers described below can be replaced or eliminated for additional embodiments of the catheter sheath 110 without departing from the scope of the present disclosure.

In some implementations, the catheter sheaths of the present disclosure are particular configured for use with PMUT or solid crystal composite rotational IVUS devices having a focused beam. In that regard, PMUT and/or other focused ultrasound transducers provide better image resolution than traditional rotational IVUS. In order to preserve the benefits of a highly focused ultrasound beam, the sheath of the catheter surrounding the rotating ultrasound transducer must not significantly distort the beam. However, the sheath must also meet the other requirements for a device suitable for introduction into a patient's body. As a result, in selecting the material properties for a sheath many, often competing, requirements must be taken into consideration. For example, it is often desirable for the material properties of the sheath to (1) minimize refraction that causes defocusing/distortion of the ultrasound beam; (2) minimize reflection that wastes energy (signal-to-noise ratio) when the ultrasound signals and/or reflections are partially reflected from a sheath interface; (3) minimize attenuation that wastes energy (signal-to-noise ratio) when the ultrasound signals and/or reflections are absorbed/dissipated by the sheath material(s); (4) minimize mode conversion that wastes energy (signal-to-noise ratio) when longitudinal waves are converted to shear waves and/or other modes at a sheath interface; (5) minimize friction with the driveshaft that spins inside the lumen of the sheath; (6) be compatible with low friction hydrophilic coating(s) to facilitate easier catheter motion inside a guiding catheter and/or the vessel of interest; and/or (7) have desired mechanical properties (e.g., flexibility, longitudinal stiffness, radial stiffness, durability, etc.) to provide the pushability, kink resistance, and/or other mechanical properties necessary for the intended use(s) of the device. As a result, the improved sheath designs of the present disclosure are particularly beneficial for use with an advanced technology focused rotational IVUS transducer to minimize distortion of the ultrasound beam while satisfying the other requirements of the IVUS catheter sheath.

As indicated above, the proximal portion 112 of the catheter sheath 110 includes a different set of material layers than the distal portion 116 of the catheter sheath 110. It is understood, however, that the catheter sheath 110 may have many different sections along its length having various combinations of material layers. In that regard, the particular combination of material layers utilized in each section is tailored to meet a desired function of that section of the intravascular device. To that end, some of the factors that dictate the type of material layers, including combinations thereof, selected for the various sections will now be discussed.

For some sections, a high lubricity, low friction material layer is desirable. In that regard, fluoropolymers (including without limitation PTFE, FEP, PFA, EFEP, and ETFE) and HDPE are generally favored for defining an inner lumen of the catheter such that the surface allows a smooth, low friction rotation of the driveshaft. An EFEP copolymer may comprise a functionalized EFEP copolymer and/or a terminally-functionalized EFEP copolymer. The chemical formula for a terminally-functionalized EFEP copolymer is:

X—(CH₂CH₂)ₘ—(CF₂CF₂)ₙ—((CF₂CF₂)CF₃)ₚ—Y,

where the letters m, n, and p represent integers. According to various embodiments, the end functional groups, —X and/or —Y, may include, but are not limited to carboxyl groups, carbonate groups, carboxyl halide groups, and/or carbonyl halide groups. An EFEP copolymer generally results from the copolymerization of tetrafluoroethylene (“TFE”), hexafluoropropylene (“HFP”), and ethylene monomers at different mole percentages via different polymerization techniques. For example, an EFEP copolymer may contain 20 to 90 mole percentage of TFE; 10 to 80 mole percentage of ethylene; and 1 to 70 mole percentage of HFP. In various embodiments, a functionalized EFEP copolymer as described above may contain, in addition to the monomer units contributed by TFE, HFP, and ethylene, one or more types of other monomers. These additional monomer(s) may be chosen such that the resulting EFEP copolymer maintains its inherent hydrophobicity. In at least one embodiment, for the convenience of melt processing during the making of a catheter shaft, for example, such EFEP copolymers may have relatively low melting points, which may be between approximately 160° C. and 240° C., as measured by a differential scanning calorimeter (“DSC”), for instance. In various embodiments, the functionalized FCP, such as terminally-functionalized EFEP copolymer, may be semi-crystalline and have a melting point lower than about 250° C., and in at least one embodiment, may have a melting point lower than about 220° C. Functionalized EFEP copolymers are available from commercial sources as NEOFLON™ RP series resins (Daikin America, Inc., Orangeburg, N.Y., USA), for example. Additional details regarding EFEP copolymers, including terminally-functionalized EFEP copolymers, and their manufacture may be found in U.S. Pat. Nos. 6,911,509 and 7,220,807, incorporated herein by reference in their entireties.

For some sections, especially the distal section where the focused ultrasound beams and reflections will propagate through, material layer(s) that provide acoustic velocities similar to the fluid in which the device is intended to be used (e.g., blood, saline, and/or other biological fluid) are desirable. Differences between sheath acoustic velocity and blood acoustic velocity cause refraction that leads to beam distortion, which can degrade the resulting image. It is difficult, if not impossible, to find a single material having the desired acoustic velocity that also satisfies all of the other necessary requirements of the sheath. Hence, a multilayered material structure is utilized in most implementations of the present disclosure. Generally, refraction is minimized if the average velocity of a multilayer sheath matches the velocity of the fluid in which the device will be used. In some instances, the layer thicknesses of the multilayer sheath are optimized beyond this first order approximation using computer simulation (e.g., using finite element analysis to calculate the optimum layer thicknesses for minimum refraction and beam distortion relative to the fluid in which the device will be used). To this end, fluoropolymers (including without limitation PTFE, FEP, PFA, EFEP, and ETFE) tend to have lower acoustic velocity than blood and saline, whereas HDPE has a higher acoustic velocity than blood and saline. SOS. Low durometer Pebax materials (e.g., 35 D, 55 D) have desirable acoustic properties as they are almost a perfect match for intravascular environments containing blood and/or saline, but such materials are “sticky” (i.e., not lubricious) and too limp for pushability. Higher durometer Pebax materials (e.g., 60 D, 70 D) are more favorable in terms of stiffness for pushability, but have less desirable acoustic properties (e.g., in the range of HDPE, in some instances).

PEBA copolymers are amine-terminated, polar polymers that comprise poly(ether) block amides. They are typically formed via polycondensation of carboxylic acid polyamides with alcohol termination polyethers. An exemplary modified or amine-terminated PEBA may include PEBAX® sold by Arkema (Colobes Cedex, France). Other suitable polymers may include VESTAMID® BS-1144 and/or BS-1145 sold by Evonik Degussa GmbH (Essen, Germany). An exemplary modified or amine-terminated polyamide may include VERSAMID® 728 sold by Cognis Corporation (Cincinnati, Ohio, USA).

Similarly, ultrasound waves are reflected by discontinuities in acoustic impedance. Sheath reflections due to discontinuities in acoustic impedance can give rise to reverberations and other undesirable image artifacts, as well as wasting energy that results in a reduced signal-to-noise ratio. An important consideration in reducing sheath reflections is minimizing the discontinuity between layers. In this regard, the goal is to keep acoustic impedance differences between the different layers as small as practical. Relative to the acoustic impedances of saline (1.5 km/s) and blood (1.6 km/s), it is desirable to keep the impedance changes to within a few tenths of a km/s (e.g., 0.4 km/s) or less if possible. Further, many polymer materials have relatively high acoustic attenuation (e.g., 3-5 dB each direction through the sheath, which is equivalent to 6-10 dB of roundtrip attenuation for the ultrasound signal), particularly at the frequencies of interest for rotational IVUS imaging (e.g., 20-80 MHz and, more particularly, 30-60 MHz). Acoustic attenuation wastes energy and, therefore, degrades the signal-to-noise ratio. Accordingly, to the extent possible, the sheath materials are designed to impart an acoustic attenuation in the desired frequency range of the ultrasound imaging to be between about 0.5 dB and about 4.0 dB for each pass through the sheath, with an obvious preference for lower attenuations. These overall acoustic attenuation values can be correlated to the thickness of the sheath in order to determine a desired attenuation per thickness value for the sheath as follows. If it is presumed that the sheath is to have a thickness of ⅛ mm and a roundtrip attenuation of 5.0 dB (2.5 dB one way) or less, then the sheath materials must collectively define an effective attenuation of 20 dB per millimeter. Similar approaches can be utilized for other sheath thicknesses and/or attenuation values.

Mode conversion from longitudinal waves to shear waves occurs whenever the ultrasound waves encounter an interface between two solids or an interface between a liquid and a solid at an angle away from perpendicular. The shear waves follow a different path from that of the longitudinal waves, creating beam distortion. Further, the shear waves tend to be more highly attenuated than the longitudinal waves in the types of polymer materials likely to be used for the acoustic window portion of the sheath. As a result, this mode conversion contributes to both beam distortion and energy waste (i.e., reduced signal-to-noise ratio). In some instances, stiffer materials with higher acoustic velocities are utilized in an effort to minimize mode conversion.

In general, the distal portion of the sheath (i.e., the portion including the acoustic window) has a thin wall to minimize distortion and attenuation. Therefore, a relatively stiff polymer is needed to provide adequate column and torsional strength for the catheter to be suitable for safe use within vessels of a patient. But stiff materials tend to have high acoustic velocity relative to blood and saline and also tend to have high acoustic impedance, which are contrary to the other desired aspects of the acoustic window.

Finally, all portions of the sheath must be made of materials of sufficient toughness and durability to prevent the sheath materials from cracking under the severe bending stress that can sometimes occur if the catheter is inadvertently prolapsed or folded within the patient's vasculature. Brittle materials that could crack under such stress may release embolic debris into the vessel that can be a moderate to severe hazard to the patient, potentially even deadly.

In FIG. 2A, the proximal portion 112 of the catheter sheath 110 includes a proximal set of material layers that provide lubricity for smooth rotation of the driveshaft, sufficient stiffness for pushability, bondability for fusing to the distal portion 114 (including the acoustic window through which ultrasound signals and reflections will propagate), kink resistance and toughness, and compatibility with hydrophilic coating(s) and/or surface treatment(s). In FIG. 2B, the distal portion 114 of the catheter sheath 110 includes a distal set of material layers that facilitates an average speed of sound through the distal set of material layers that is substantially equivalent to a speed of sound through blood, thereby minimizing image distortion resulting from beam distortion of the ultrasound signals traveling through the distal portion 114 of the catheter sheath 110.

The particular combinations of material layers used for the proximal and distal portions of the catheter sheath are selected based on the various parameters discussed above. Accordingly, in some instances, the proximal portion 112 and/or the distal portion 114 includes an inner layer of HDPE, FEP, PTFE, PFA, ETFE, and/or other fluoropolymer that defines an inner lumen of the sheath that provides low friction. Unfortunately, many of these materials are difficult to bond to. Further, fluoropolymers tend to have low acoustic velocities relative to blood and saline. Therefore, in the distal portion 114 of the sheath where acoustic properties are important, the low friction, low velocity, difficult to bond with fluoropolymer inner layer is preferably paired with an outer layer having a higher acoustic velocity in order to produce an average sheath velocity close to that of blood/saline that results in a low refraction/beam distortion. Furthermore, in some instances the outer layer is readily bondable to other components of the sheath including the proximal segment, distal tip, and/or inner layer. To facilitate bonding the outer layer to the inner layer, an intermediate tie layer is included in some instances. In addition to or in lieu of the intermediate tie layer, the inner layer may be etched, plasma-treated, or otherwise modified to more readily bond to the outer layer. Choosing the materials to provide an average speed of sound close to that of saline/blood is a first order approximation to minimize refraction/distortion of the ultrasound beam. However, as noted above, a more optimal acoustic design can be found through finite element analysis of acoustic wave propagation through the sheath taking into account the material properties and structural design. Besides minimizing beam distortion due to refraction, consideration is also given to total internal reflection (cutoff) at the interface from a slower material to a faster material, as well as mode conversion from longitudinal waves into shear waves or other modes that mostly represent lost signal or increased attenuation with corresponding reduction in signal-to-noise ratio.

While some specific embodiments are discussed herein, various embodiments of the proximal set of material layers and the distal set of material layers that achieve the characteristics described herein include polytetrafluoroethylene (PTFE), ethylene tetrafluoroethylene (ETFE), fluorinated ethylene propylene (FEP), expanded fluorinated ethylene propylene (EFEP), polyether block amide (PEBA, for example available under the trade name PEBAX®), biocompatible polymers, and/or other materials that achieve the characteristics of the proximal portion 112 and the distal portion 116 described herein, or combinations thereof. It is noted that, in the depicted embodiment, the proximal portion 112 has a diameter or thickness, T₁, that is greater than a diameter or thickness, T₂, of the distal portion 116. In some embodiments, the proximal portion 112 and the distal portion 116 have a same thickness, or the proximal portion 112 has a thickness less than the distal portion 116.

In FIGS. 2A and 2B, the proximal portion 112 of the catheter sheath 110 has an inner surface (diameter) 150 and an outer surface (diameter) 152, and the distal portion 116 of the catheter sheath 110 has an inner surface (diameter) 154 and an outer surface (diameter) 156. The inner surfaces 150 and 154 have a coefficient of friction with respect to the imaging core 120 that minimizes friction between the imaging core 120 and the catheter sheath 110. Minimizing friction between the imaging core 120 and the inner surfaces 150, 154 of the catheter sheath 110 allows the core to rotate more freely. In some embodiments, the inner surfaces 150 and 154 have a low coefficient of friction (μ). The coefficient of friction is a dimensionless scalar value that describes the ratio of the force of friction between two bodies and the force pressing them together. Because it is a two body measurement, coefficient of friction is typically indexed with respect to a common test material, such as steel or glass. Polyethylene, commonly used in catheters, has a coefficient of friction against steel of 0.2. Fluoropolymers suitable for use as the inner surface 150, typically have a coefficient of friction against steel of 0.1 or less. PTFE, which may be used for the inner surface 150 in some instances, has a coefficient of friction against steel of 0.04. EFEP, used as the inner surface 154 in some instances, has a coefficient of friction against steel of 0.06. Blends of fluoropolymers and stiffer polymers such as polyimides and polyamides may also be used to provide better push characteristics in the catheter. In one embodiment, an inner polymer layer of the proximal portion of the catheter includes a polyimide/PTFE blend that has a coefficient of friction of 0.07 versus steel.

The outer surface 152 and/or outer surface 156 have a surface energy that facilitates application of a hydrophilic coating 158 on the outer surface 152 and/or the outer surface 156. In some embodiments, the outer surfaces 152 and 156 have a surface energy of between about 20 dynes/cm² and about 60 dynes/cm². In the depicted embodiment, the outer surfaces 152 and 156 have a surface energy greater than a surface energy of a catheter sheath having an outer surface formed by a polyethylene material, such as about 45 dynes/cm². The hydrophilic coating 158 on the outer surface 152 and/or the outer surface 156 reduces friction between the catheter sheath 110 and the inner surface of the guiding catheter and between the catheter sheath and the vessel lumen that the catheter sheath 110 contacts while in use. The hydrophilic coating 158 also draws blood to the outer surface 156, thereby providing wetting between blood in the vasculature and the distal portion 116 of the catheter sheath 110. It is noted that, because polyethylene has a low surface energy and poor wetting with respect to blood, hydrophilic coatings cannot be readily applied to conventional polyethylene catheters.

In the present example, referring to FIGS. 2A and 2B, the proximal set of material layers includes a proximal outer layer 160 (defining outer surface 152), a proximal intermediate layer 162, and a proximal inner layer 164 (defining inner surface 150); and the distal set of material layers include a distal outer layer 170 (defining outer surface 156) and a distal inner layer 172 (defining inner surface 154). Other embodiments may include more or fewer layers in the proximal set of material layers and/or the distal set of material layers. The proximal outer layer 160 and the distal outer layer 170 include a material, such as PEBAX®, that exhibits a surface energy that facilitates application of the hydrophilic coating 158 on the outer surface 152 and/or the outer surface 156 of the catheter sheath 110, as described above. In the depicted embodiment, the proximal outer layer 160 and the distal outer layer 170 include a same material, which facilitates coupling of the proximal portion 112 and the distal portion 116 of the catheter sheath 110. For example, the proximal outer layer 160 and the distal outer layer 170 are thermally fused together so that the proximal portion 112 is physically coupled with the distal portion 116. Alternatively, the proximal outer layer 160 and the distal outer layer 170 include different materials that are assembled together (e.g., by thermally fusing the same material or compatible materials together and/or gluing or mechanically coupling) to couple the proximal portion 112 to the distal portion 116.

The proximal intermediate layer 162 includes a material that provides strength to the proximal portion 112 of the catheter sheath 110. In some embodiments, the proximal intermediate layer 162 includes a metal, such as stainless steel, superelastic material such as Nitinol, high-strength polymer fibers (e.g., carbon-fiber, Spectra (polyethylene fiber), Kevlar, Dacron), and/or combinations thereof. In the present example, the proximal intermediate layer 162 includes a metal braid layer that provides strength to the proximal portion 112 of the catheter sheath 110, such as a stainless steel braid formed in polyimide (SS Wire Braid/PI). The metal braid layer includes divots (not shown) in which a material of the proximal inner layer 160 is formed, such that a proximal inner layer 170 formed of a substantially constant thickness includes corresponding divots, thereby minimizing contact area between the proximal inner layer 170 and the imaging core 120 during use.

The proximal inner layer 160 defines the lumen 122 in the proximal portion 112 of the catheter sheath 110. The proximal inner layer 160 includes a material having a coefficient of friction that minimizes friction between the imaging core 120 and the catheter sheath 110, as described above. Minimizing friction between the imaging core 120 and the catheter sheath 110 allows the imaging core to more freely rotate within the catheter sheath. In some embodiments, the material of the proximal inner layer 160 has a coefficient of friction (μ) between of 0.1 or less. In the depicted embodiment, the proximal inner layer 160 includes a material having a static coefficient of friction of about 0.07 and a kinetic coefficient of friction of about 0.13. For example, the proximal inner layer 160 includes a polymer blend, such as a PI/PTFE polymer blend.

The distal inner layer 172 defines the lumen 122 in the distal portion 116 of the catheter sheath 110. The distal inner layer 172 includes a material having a coefficient of friction that minimizes friction between the imaging core 120 and the catheter sheath 110, as described above, where the material also facilitates an average speed of sound through the distal portion 116 of the catheter sheath 110 that is substantially equivalent to a speed of sound through blood. Minimizing friction between the imaging core 120 and the catheter sheath 110 allows the imaging core to more freely rotate within the catheter sheath, and ensuring that the speed of sound through the distal portion 116 is substantially equivalent to the speed of sound through blood minimizes ultrasound signal distortion. In some embodiments, an average speed of sound through the distal inner layer 172 is about 1.40 km/s to about 1.70 km/s. In the depicted embodiment, for example, the distal inner layer 172 includes EFEP, through which a speed of sound is about 1.40 km/s. In some embodiments, the distal inner layer 172 includes other materials that facilitate the speed of sound through the distal portion 116 being substantially equivalent to the speed of sound through blood, such as PEBAX 4033, EVA/Ve-634 (28% acetate) (about 1.67 km/s and about 1.68 km/s, respectively), or a combination thereof.

As noted above, the distal set of material layers of the distal portion 116 facilitate an average speed of sound through the distal set of material layers that is substantially equivalent to a speed of sound through blood, thereby minimizing image distortion resulting from the ultrasound signals traveling through the distal portion 116 of the catheter sheath 110. In some embodiments, an average speed of sound through the distal set of material layers is about 1.50 km/s to about 1.60 km/s. In some embodiments, an average speed of sound through the distal set of material layers is about 1.52 km/s. In the present example, the distal outer layer 170 includes the polyether block amide material, such as PEBAX®, and the distal inner layer 172 includes the EFEP material. A speed of sound through polyether block amide material, such as PEBAX®, varies with its hardness. Accordingly, in furtherance of the present example, polyether block amide material, such as PEBAX®, has a hardness of about 72 D (durometer), where a speed of sound through such material is about 1.99 km/s, and the speed of sound through the EFEP material is about 1.40 km/s. By providing the distal set of material layers with about 75% EFEP material and about 25% polyether block amide material, an average speed of sound through the distal portion 116 of the catheter sheath 110 is about 1.55 km/s. As a more general example, for a two layer sheath having a thickness T, if the first material layer comprises 75% of the thickness of the sheath (i.e., 0.75 T) and has an acoustic velocity V₁ and the second material layer comprises 25% of the thickness of the sheath (i.e., 0.25 T) and has an acoustic velocity V₂, then average acoustic velocity of the sheath can be calculated as V_{avg}=0.75*V₁+0.25*V₂ or other suitable formulaic representation. This approach can be extended to any number of material layers. It is noted that, in some embodiments, the lumen 122 is filled with a saline-type material, where an average speed of sound through the saline-type material is substantially equivalent to the speed of sound through blood.

From the foregoing, the disclosed catheter sheath 110 includes combinations of materials in the proximal portion 112 and the distal portion 116 that minimize ultrasound signal distortion, while providing sufficient strength and flexibility for use within human vasculature. For example, the materials of the catheter sheath 110 minimize friction between the catheter sheath 110 and the imaging core 120, minimize friction between the catheter sheath 110 and the vasculature along its path to the vessel of interest, enable application of a hydrophilic coating on the outer surface of the catheter sheath 110, provide sufficient strength and flexibility, and/or facilitate travel of sound through the catheter sheath 110 similar to travel of sound through blood. It is noted that thicknesses of the material layers in the distal portion 116 and the proximal portion 120 may be varied to achieve the desired characteristics and optimize the catheter's minimal contribution to image distortion. Different embodiments may have different advantages, and no advantage is necessarily required of any embodiment.

Various methods may be employed to produce a catheter having the properties discussed above. In various embodiments, a melt process, such as mono-extrusion, sequential extrusion, co-extrusion, and/or heat lamination (reflow), and the like may be utilized to produce the proximal and/or distal portions of the catheter. In more detail, two such catheter manufacturing processes are sequential mono-extrusion and heat lamination, or reflow. In the sequential mono-extrusion shaft manufacturing process, the inner polymeric layer is first extruded over a continuous, supportive core rod having a melting temperature higher than that of the extrusion temperature of the layer. Next, the outer polymeric layer is over-extruded onto the inner polymer layer. Various manufacturers can provide co-extrusion tubing to be used in the shafts of the invention, such as Teleflex Medical OEM (Limerick, Ireland). A sequential extrusion or a sequential mono-extrusion technique may also be utilized. In such an embodiment, a first polymer, such as EFEP, is extruded to form the inner layer and a second polymer, such as PEBA is then extruded onto the inner layer to form the second polymer layer. The mono-extrusion process may be carried out with a regular single-screw extruder. A hydrophilic barrier layer may then be formed on the outer layer after the extrusion is completed. The heat energy of the extrusion process and/or the reflow/heat lamination process, if such is applied, may assist in forming such a direct and/or covalent bond between the outer layer and inner layer.

Further, as noted above, in some implementations the proximal portion of the catheter includes reinforcing elements, such as braids, wires, cages, coils, hoops, or helixes formed of a suitable material. In such instances, the inner polymeric layer may be first extruded (as discussed above) and then the reinforcing element(s) may be formed by braiding strands of material, for example, onto the inner polymeric layer. In such an embodiment, the outer polymeric layer may be formed by extruding a polymer, such as an amine-terminated PEBA, for example, over the reinforcing element(s) and the inner polymeric layer. Once completed, the outer polymer layer may be coated with another polymer or coating to provide a hydrophilic outer surface.

Alternatively, in a reflow manufacturing process where the inner and outer polymeric layers may be are prepared via polymer extrusion processes, a pre-made reinforcing element (e.g., in a given weaving pattern made via braiding) is provided separately. The inner layer, the reinforcing element, and the outer layer are then layer-by-layer introduced onto a supportive, metallic core rod and incorporated into a single, cylindrical, shaft body via a heat lamination, or reflow, processes by applying an external heat source over a proper shrink tube that completely and circumferentially embraces the shaft body to be formed. In some instances, this process results in a more continuous axial transition from the inner polymer layer to the outer polymer layer due to the pressure and heat. The inner and outer polymer layers may largely contain the reinforcing element there between and, ideally, be bonded onto the reinforcement element and/or to the other layer through the reinforcement element. As such, the contained reinforcement element of the bonded polymeric layers may provide some reinforcing effects for the shaft body in terms of column strength, fracture energy, and/or kink resistance, and the like. Additionally, using this process the interior ridges or projections associated with the shape of the reinforcement element are more pronounced, which results in even less contact surface area on the interior of the proximal portion of the catheter. Proximal shaft tubing fabricated with either process is available from commercial suppliers, such as Teleflex Medical OEM.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An intravascular ultrasound (IVUS) device comprising:
a flexible elongate member having a lumen extending therethrough, wherein the flexible elongate member has a proximal portion coupled with a distal portion;
an imaging core disposed within the lumen, the imaging core configured to rotate within the lumen and further configured to transmit a focused ultrasound signal and receive ultrasound echoes through the distal portion of the flexible elongate member; and
wherein:
the distal portion includes a first set of material layers that facilitates an average speed of sound through the first set of material layers that is substantially equivalent to a speed of sound through blood, and
the proximal portion includes a second set of material layers different than the first set of material layers,
wherein at least the first set of material layers is configured to minimize distortion of the focused ultrasound signal transmitted through the distal portion of the flexible elongate member and minimize distortion of the received ultrasound echoes.

2. The IVUS device of claim 1 wherein the imaging core includes a transducer assembly coupled to the distal portion of the flexible elongate member.

3. The IVUS device of claim 2 wherein the transducer assembly includes a piezoelectric micromachined ultrasound transducer (PMUT).

4. The IVUS device of claim 1 wherein the average speed of sound through the first set of material layers is between about 1.50 km/s and about 1.60 km/s.

5. The IVUS device of claim 1 wherein the distal portion and the proximal portion have outer surfaces having a surface energy that is greater than a surface energy of polyethylene.

6. The IVUS device of claim 6 wherein the surface energy is between about 20 dynes/cm² and about 60 dynes/cm².

7. The IVUS device of claim 1 further comprising a hydrophilic coating disposed on an outer surface of the distal portion of the flexible elongate member.

8. The IVUS device of claim 7 wherein the hydrophilic coating is further disposed on an outer surface of the proximal portion of the flexible elongate member.

9. The IVUS device of claim 1 wherein the lumen is configured to be filled with a saline-type material.

10. The IVUS device of claim 9 wherein the saline-type material facilitates an average speed of sound through the saline-type material that is substantially equivalent to the speed of sound through blood.

11. The IVUS device of claim 1 wherein the first set of material layers and the second set of material layers include at least one of a polyether blockamide material, an expanded fluorinated ethylene propylene material, a polytetrafluoroethylene material, a polyimide material, a metal material, and a combination thereof.

12. An ultrasound catheter comprising:
a flexible elongate member having a lumen extending therethrough, wherein the flexible elongate member has a proximal portion physically coupled with a distal portion, wherein:
the distal portion includes a distal outer layer and a distal inner layer, wherein an average speed of sound through the distal portion is substantially equivalent to a speed of sound through blood; and
the proximal portion includes a proximal outer layer, a proximal inner layer, and a proximal intermediate layer disposed between the proximal outer layer and the proximal inner layer.

13. The ultrasound catheter of claim 12 wherein the distal outer layer and the proximal outer layer are formed of the same material.

14. The ultrasound catheter of claim 13 wherein the distal outer layer and the proximal outer layer are formed of a polyether blockamide material.

15. The ultrasound catheter of claim 13 wherein the distal outer layer and the proximal outer layer include a material having a surface energy that is greater than a surface energy of polyethylene.

16. The ultrasound catheter of claim 12 wherein the average speed of sound through the distal portion is between about 1.50 km/s and about 1.60 km/s.

17. The ultrasound catheter of claim 16 wherein the distal inner layer includes a material having an average speed of sound therethrough between about 1.40 km/s and about 1.70 km/s.

18. The ultrasound catheter of claim 16 wherein:
the distal outer layer includes a polyether blockamide material; and
the distal inner layer includes an expanded fluorinated ethylene propylene material.

19. The ultrasound catheter of claim 12 further including a hydrophilic coating disposed on the distal outer layer and the proximal outer layer.

20. The ultrasound catheter of claim 12 wherein the proximal intermediate layer includes a metal braid layer.

21. The ultrasound catheter of claim 12 wherein:
the distal outer layer includes a polyether blockamide material;
the distal inner layer includes an expanded fluorinated ethylene propylene material;
the proximal outer layer includes the polyether blockamide material;
the proximal intermediate layer includes a stainless steel wire braid material;
the proximal inner layer includes a blend of polytetrafluoroethylene and polyimide.

22. An intravascular ultrasound (IVUS) system comprising:
an imaging device that includes:
a flexible elongate member having a lumen extending therethrough, wherein the flexible elongate member has a proximal portion and a distal portion, and further wherein an average speed of sound through the distal portion is substantially equivalent to a speed of sound through blood, and
an imaging core disposed within the lumen, the imaging core configured to rotate within the lumen and further configured to transmit and receive ultrasound signals through the distal portion of the flexible elongate member; and
an interface module configured to engage with the proximal portion of the flexible elongate member; and
an image processing component in communication with the interface module.

23. The IVUS system of claim 22 wherein the average speed of sound through the distal portion is between about 1.50 km/s and about 1.60 km/s.
